SEQUENCE LISTING (21 sequences)

SEQ ID NO: 1 - protein, 20 aa, Artificial Sequence
  Description of Artificial Sequence Synthetic peptide
MDAMKRGLCCVLLLCGAVFV

SEQ ID NO: 2 - DNA, 60 bp, Artificial Sequence
  Description of Artificial Sequence Synthetic oligonucleotide
  CDS (1)..(60)
atggatgcaatgaagaggggcctgtgctgcgtgctgctgctgtgtggcgccgtgtttgtg

SEQ ID NO: 3 - DNA, 60 bp, Artificial Sequence
  Description of Artificial Sequence Synthetic oligonucleotide
atggatgccatgaagagaggactgtgctgcgtgctgctgctctgtggagccgtctttgtg

SEQ ID NO: 4 - protein, 20 aa, Artificial Sequence
  Description of Artificial Sequence Synthetic peptide
SPSEITRAPRCPAVRSLLRS

SEQ ID NO: 5 - DNA, 60 bp, Artificial Sequence
  Description of Artificial Sequence Synthetic oligonucleotide
  CDS (1)..(60)
agccctagcgagatcaccagagcccccagatgccctgccgtgagaagcctgctgcggagc

SEQ ID NO: 6 - DNA, 60 bp, Artificial Sequence
  Description of Artificial Sequence Synthetic oligonucleotide
agccctagcgagatcacccgggctcccagatgccctgccgtccggagcctgctgcggagc

SEQ ID NO: 7 - DNA, 35 bp, Artificial Sequence
  Description of Artificial Sequence Synthetic oligonucleotide
ctagtattcccctgacccgccataacaggcccggc

SEQ ID NO: 8 - DNA, 28 bp, Artificial Sequence
  Description of Artificial Sequence Synthetic oligonucleotide
ctcatggttacccaggcgaagccaggta

SEQ ID NO: 9 - DNA, 3798 bp, Artificial Sequence
  Description of Artificial Sequence Synthetic polynucleotide
agatctgatatcgccaccatggatgcaatgaagaggggcctgtgctgcgtgctgctgctgtgtggcgccgtgtttgtgagccctagcgagatcaccagagcccccagatgccctgccgtgagaagcctgctgcggagccggtacagagaagtgtggcccctggccacctttgtgaggagactgggccctgagggcaggagactggtgcagcctggcgaccccaaaatctacaggaccctggtggcccagtgtctggtgtgtatgcactggggcagccagccccctcccgccgacctgagcttccaccaggtgtccagcctgaaggaactggtggccagagtggtgcagagactgtgcgagcggaacgagagaaacgtgctggccttcggcttcgagctgctgaacgaggccagaggcggccctcccatggccttcaccagctctgtgaggagctacctgcccaacaccgtgatcgagaccctgagagtgagcggcgcctggatgctgctgctgagcagagtgggcgatgacctgctggtgtacctgctggcccactgcgccctgtatctgctggtgccccccagctgcgcctaccaggtgtgcggatcccccctgtaccagatttgcgccaccaccgacatctggcccagcgtgtctgccagctacagacccaccagacctgtgggccggaacttcaccaacctgcggttcctgcagcagatcaagagcagcagcagacaggaggcccccaagcccctggccctgcccagcagaggcaccaagagacacctgagcctgaccagcaccagcgtgcccagcgccaagaaagccagatgctaccccgtgcctagagtggaggagggccctcacagacaggtgctgcccacccccagcggcaagagctgggtgcccagccccgccagaagccccgaagtgcccaccgccgagaaggacctgagcagcaagggcaaagtgagcgacctgtctctgagcggcagcgtgtgttgcaagcacaagcccagcagcaccagcctgctgagcccccccagacagaacgccttccagctgaggcctttcatcgagacccggcacttcctgtacagcagaggcgatggccaggagagactgaaccccagcttcctgctgagcaacctgcagcctaacctgaccggcgccagacgcctggtggagatcatcttcctgggcagcagacccagaaccagcggccctctgtgcagaacccaccggctgagcaggcggtactggcagatgagacccctgttccagcagctgctggtgaaccacgccgagtgccagtatgtgcggctgctgaggagccactgcagattcaggaccgccaaccagcaggtgaccgacgccctgaacaccagcccccctcacctgatggatctgctgaggctgcacagcagcccctggcaggtgtacggcttcctgagagcctgcctgtgcaaagtggtgtccgccagcctgtggggcaccagacacaacgagcggcggttcttcaagaatctgaagaagttcatcagcctgggcaagtacggcaagctgagcctgcaggaactgatgtggaagatgaaagtggaggactgccactggctgagaagcagccccggcaaggacagagtgcctgccgccgagcacagactgagggagagaatcctggccacattcctgttctggctgatggacacctacgtggtgcagctgctgcggtccttcttctacatcaccgagagcaccttccagaagaaccggctgttcttctaccggaagtctgtgtggagcaagctgcagagcatcggagtgagacagcacctggagagagtgaggctgagagagctgagccaggaggaagtgagacaccaccaggatacctggctggccatgcccatctgccggctgagattcatccccaagcccaacggcctgagacccatcgtgaacatgagctacagcatgggcacaagagccctgggcagaagaaagcaggcccagcacttcacccagcggctgaaaaccctgttctccatgctgaactacgagcggaccaagcacccacacctgatgggcagcagcgtgctgggcatgaacgacatctaccggacctggagagccttcgtgctgagagtgcgggccctggaccagacccctcggatgtacttcgtgaaggccgccatcaccggcgcctacgacgccatcccccagggcaaactggtggaagtggtggccaacatgatcaggcacagcgagtccacctactgcatcaggcagtacgccgtggtgagaagagacagccagggccaggtgcacaagagcttccggagacaggtgaccaccctgagcgatctgcagccttacatgggccagttcctgaagcacctgcaggatagcgacgccagcgccctgagaaatagcgtggtgatcgagcagagcatcagcatgaacgagtccagcagcagcctgttcgacttcttcctgcacttcctgaggcacagcgtggtgaagatcggcgacagatgctacacccagtgtcagggcatccctcagggctctagcctgagcaccctgctgtgtagcctgtgcttcggcgacatggagaataagctgttcgccgaagtgcagagagatggcctgctgctgcgcttcgtggacgatttcctgctggtgaccccacacctggaccaggccaagaccttcctgagcacactggtgcacggcgtgcccgagtacggctgcatgatcaatctgcagaaaaccgtggtgaacttccctgtggagcccggcaccctgggcggagccgccccttaccagctgcccgcccactgcctgttcccctggtgcggactgctgctggatacccagaccctggaagtgttctgcgactacagcggctacgcccagaccagcatcaagaccagcctgaccttccagagcgtgttcaaggccggcaagaccatgaggaacaagctgctgagcgtgctgagactgaagtgccacggcctgttcctggatctgcaggtgaacagcctgcagaccgtgtgtatcaacatctacaagattttcctgctgcaggcctacagattccacgcctgcgtgatccagctgcccttcgaccagagagtgcggaagaacctgaccttcttcctggggatcatcagcagccaggccagctgctgctacgccatcctgaaagtgaagaaccccggcatgaccctgaaggccagcggcagcttccctcccgaggccgcccactggctgtgctaccaggcctttctgctgaagctggccgcccacagcgtgatctacaagtgcctgctgggccctctgagaaccgcccagaagctgctgtgccggaagctgcccgaggccaccatgaccattctgaaagccgccgccgaccccgccctgagcaccgacttccagaccatcctggactctagagcccctcagagcatcaccgagctgtgcagcgagtaccggaacacccagatttacaccatcaacgacaagatcctgagctacaccgagtctatggccggcaagcgggagatggtgatcatcaccttcaagagcggcgccacctttcaggtggaagtgcctggcagccagcacatcgacagccagaagaaggccatcgagcggatgaaggacaccctgcggatcacctacctgaccgagaccaagatcgacaagctgtgtgtgtggaacaacaagacccccaacagcatcgccgccatctctatggagaactgatctagaaattaagtcgacgaattc

SEQ ID NO: 10 - protein, 1251 aa, Artificial Sequence
  Description of Artificial Sequence Synthetic polypeptide
MDAMKRGLCCVLLLCGAVFVSPSEITRAPRCPAVRSLLRSRYREVWPLATFVRRLGPEGRRLVQPGDPKIYRTLVAQCLVCMHWGSQPPPADLSFHQVSSLKELVARVVQRLCERNERNVLAFGFELLNEARGGPPMAFTSSVRSYLPNTVIETLRVSGAWMLLLSRVGDDLLVYLLAHCALYLLVPPSCAYQVCGSPLYQICATTDIWPSVSASYRPTRPVGRNFTNLRFLQQIKSSSRQEAPKPLALPSRGTKRHLSLTSTSVPSAKKARCYPVPRVEEGPHRQVLPTPSGKSWVPSPARSPEVPTAEKDLSSKGKVSDLSLSGSVCCKHKPSSTSLLSPPRQNAFQLRPFIETRHFLYSRGDGQERLNPSFLLSNLQPNLTGARRLVEIIFLGSRPRTSGPLCRTHRLSRRYWQMRPLFQQLLVNHAECQYVRLLRSHCRFRTANQQVTDALNTSPPHLMDLLRLHSSPWQVYGFLRACLCKVVSASLWGTRHNERRFFKNLKKFISLGKYGKLSLQELMWKMKVEDCHWLRSSPGKDRVPAAEHRLRERILATFLFWLMDTYVVQLLRSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKAAITGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILDSRAPQSITELCSEYRNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 11 - protein, 253 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLRGSHHHHHHAAAS

SEQ ID NO: 12 - DNA, 765 bp, Artificial Sequence
  Description of Artificial Sequence Synthetic polynucleotide
  CDS (4)..(762)
catatggtgtccaaaggcgaagaactgttcaccggcgtggtgccgattctggtggaactggatggcgatgtgaacggccacaaattcagcgtgtccggcgaaggtgaaggtgatgccacctacggcaaactgaccctgaaattcatctgtaccaccggcaaactgccggtgccgtggccgaccctggtgaccaccctgacctacggcgtgcagtgcttctctcgctacccggatcacatgaaacagcacgatttcttcaaaagcgccatgccggaaggctacgtgcaggaacgtaccattttcttcaaagatgatggcaactacaaaacccgtgccgaagtgaaattcgaaggcgataccctggtgaaccgtatcgaactgaaaggcatcgactttaaagaggacggtaacatcctgggccacaaactggaatacaactacaacagccacaacgtgtacatcatggccgataaacagaaaaacggcatcaaagtgaacttcaaaatccgccacaacatcgaagatggcagcgtgcagctggccgatcactaccagcagaacaccccgattggtgatggcccggtgctgctgccggataaccactacctgagcacccagagcgccctgagcaaagatccgaacgaaaaacgtgatcacatggtgctgctggaattcgtgaccgccgctggtattaccctgggcatggatgaactgtacaagcttagaggatctcaccatcaccatcaccatgcggccgcatcgtga

SEQ ID NO: 13 - DNA, 765 bp, Artificial Sequence
  Description of Artificial Sequence Synthetic polynucleotide
catatggtgagtaaaggtgaagaattattcacgggcgtggttccaattctggttgaactggatggcgatgtgaacggtcacaaattcagtgttagcggcgaaggcgaaggtgatgcgacgtacggcaaactgacgctgaaattcatctgtaccaccggcaaactgccggttccatggccgacgctggttacgaccttaacctacggcgttcagtgcttcagtcgttacccagatcacatgaaacagcacgatttcttcaaaagcgccatgccagaaggttacgttcaggaacgtacgattttcttcaaagatgatggcaactacaaaacccgtgcggaagtgaaattcgaaggtgataccttagtgaaccgtatcgaattaaaaggcatcgactttaaagaggacggcaacatcttaggtcacaaattagaatacaactacaacagccacaacgtgtacatcatggcggataaacagaaaaacggcatcaaagttaacttcaaaatccgccacaacatcgaagatggtagtgtgcagttagcggatcactaccagcagaacaccccgattggcgatggcccggttttactgccagataaccactacctgagtacccagagtgccctgagcaaagatccaaacgaaaaacgtgatcacatggttttactggaattcgttacggcggcgggcattacgctgggcatggatgaactgtacaagcttagaggatctcaccatcaccatcaccatgcggccgcatcgtga

SEQ ID NO: 14 - protein, 250 aa, Artificial Sequence
  Description of Artificial Sequence Synthetic polypeptide
MAAPSDGFKPRERSGGEQAQDWDALPPKRPRLGAGNKIGGRRLIVVLEGASLETVKVGKTYELLNCDKHKSILLKNGRDPGEARPDITHQSLLMLMDSPLNRAGLLQVYIHTQKNVLIEVNPQTRIPRTFDRFCGLMVQLLHKLSVRAADGPQKLLKVIKNPVSDHFPVGCMKVGTSFSIPVVSDVRELVPSSDPIVFVVGAFAHGKVSVEYTEKMVSISNYPLSAALTCAKLTTAFEEVWGVIHHHHHH

SEQ ID NO: 15 - DNA, 764 bp, Artificial Sequence
  Description of Artificial Sequence Synthetic polynucleotide
  CDS (3)..(752)
ccatggctgctcctagcgacggcttcaagccccgggagcggagcggcggagagcaggcccaggactgggacgccctgccccccaagcggcctagactgggagccggcaacaagatcggcggcaggcggctgatcgtggtgctggaaggcgccagcctggaaaccgtgaaagtgggcaagacctacgagctgctgaactgcgacaagcacaagagcatcctgctgaagaacggccgggaccccggcgaggccaggcccgacatcacccaccagagcctgctgatgctcatggattcccccctgaacagagccggcctgctgcaggtgtacatccacacccagaaaaacgtgctgatcgaggtgaacccccagaccagaatcccccggaccttcgaccggttctgcggcctgatggtccagctgctccataagctgtccgtgagagccgccgacggcccccagaaactgctgaaggtgatcaagaaccccgtgagcgaccacttccccgtgggctgcatgaaagtggggaccagcttcagcatccccgtggtgtccgacgtgcgggagctggtgcccagcagcgaccccatcgtgttcgtggtgggcgccttcgcccacggcaaggtgtccgtggagtacaccgagaagatggtgtccatcagcaactaccccctgtctgccgccctgacctgcgccaagctgaccaccgccttcgaggaagtgtggggcgtgatccaccaccaccaccaccactgataactcgag

SEQ ID NO: 16 - DNA, 764 bp, Artificial Sequence
  Description of Artificial Sequence Synthetic polynucleotide
ccatggccgctcctagcgacggcttcaagcccagagagcgctccggcggagagcaggcccaggactgggacgccctcccccccaagagacctagactcggagccggaaacaagatcggcggcaggaggctcatcgtcgtgctggaaggcgcttccctggaaacagtgaaagtgggaaagacctacgagttgctcaactgcgacaagcacaagtccatcctcctcaagaacggaagggaccctggcgaggctaggcctgacatcacacaccagagcctgctcatgctcatggatagccccctgaacagggctggactcctccaggtctacatccacacccagaaaaacgtgctcatcgaggtcaaccctcagacaagaatccctaggacattcgacaggttctgcggcctgatggtgcagctcctgcataagctctccgtcagggctgctgacggacctcagaaactgctgaaggtcatcaagaaccccgtcagcgaccacttccccgtgggatgcatgaaagtcggcacctcattcagcatccctgtcgtcagcgacgtcagagagttggtcccctcctccgaccccatcgtcttcgtcgtgggcgctttcgcccacggaaaggtgtccgtcgagtacacagagaagatggtgtccatcagcaactaccctctgtccgccgctctgacctgcgctaagctcaccacagccttcgaggaagtgtggggcgtgatccaccaccaccaccaccactgataactcgag

SEQ ID NO: 17 - DNA, 764 bp, Artificial Sequence
  Description of Artificial Sequence Synthetic polynucleotide
ccatggctgccccctccgacggcttcaagcctagagagaggagcggaggggagcaggctcaggactgggacgccctgcctcctaagaggcccagactgggagccggcaacaagatcggcggcaggaggctgatcgttgtcctcgaaggagctagcctggaaacagtgaaagtcggaaagacctacgagctgctgaactgcgacaagcacaagtccatcctcctcaagaacggcagggaccccggcgaggctaggcccgacatcacacaccagtccctgctgatgctgatggattcccctctgaacagggctggactgctccaggtgtacatccacacacagaaaaacgtcctcatcgaggttaaccctcagacaaggatccccaggaccttcgacaggttctgcggactgatggtgcagctgctccataagctcagcgtcagggctgctgacggcccccagaaactcctcaaagtcatcaagaaccccgttagcgaccacttccccgtgggctgcatgaaagtcggaacaagcttctccatccctgttgtcagcgacgtcagggagttggtgcctagctccgaccccatcgtgttcgtcgtcggagctttcgcccacggaaaagttagcgtggagtacaccgagaagatggtctccatcagcaactaccccctgtccgcagccctcacctgcgccaagctgacaaccgctttcgaggaagtgtggggcgtgatccaccaccaccaccaccactgataactcgag

SEQ ID NO: 18 - protein, 6 aa, Artificial Sequence
  Description of Artificial Sequence Synthetic 6xHis tag
HHHHHH

SEQ ID NO: 19 - protein, 5 aa, Artificial Sequence
  Description of Artificial Sequence Synthetic peptide
VPTAG

SEQ ID NO: 20 - protein, 10 aa, Artificial Sequence
  Description of Artificial Sequence Synthetic peptide
DEKNIQHCYF

SEQ ID NO: 21 - protein, 18 aa, Artificial Sequence
  Description of Artificial Sequence Synthetic peptide
GEDAVRSKNTIQHPLCYF

(19) United States
(12) Patent Application Publication
(10) Pub. No.: US 2018/0086781 A1
(43) Pub. Date: Mar. 29, 2018

(54) STEGANOGRAPHIC EMBEDDING OF INFORMATION IN CODING GENES

(71) Applicant: GENEART AG, Carlsbad, CA (US)

(72) Inventor: Michael LISS, Regensburg (DE)

(21) Appl. No.: 15/673,541

(22) Filed: Aug. 10, 2017

Related U.S. Application Data
(62) Division of application No. 14/340,550, filed on Jul. 24, 2014, now abandoned.
Application No. 14/340,550 is a division of application No. 12/745,204, filed on Dec. 14, 2010, now abandoned.

(30) Foreign Application Priority Data
Nov. 30, 2007 (DE) 102007057802.6

(51) Int. Cl. C07H 1/00; H04L 9/08; C12N 15/63; C07H 21/04; G06F 19/22; C12Q 1/68

(52) U.S. Cl. CPC C07H 1/00; H04L 2209/24; H04L 9/0816; C12N 15/63; C12Q 2563/185; G06F 19/22; C12Q 1/68; C07H 21/04

(57) ABSTRACT

The present invention relates to the storage of information in nucleic acid sequences. The invention also relates to nucleic acid sequences containing desired information and to the design, production or use of sequences of this type.

### CROSS-REFERENCE TO RELATED APPLICATIONS

This application is a divisional of U.S. application Ser. No. 14/340,550 filed Jul. 24, 2014, now pending, which is a divisional of U.S. application Ser. No. 12/745,204 filed on Dec. 14, 2010, now abandoned, which is a 371 Application of International Application PCT/EP2008/010128 filed on Nov. 28, 2008, and claims priority to German application no. 102007057802.6, filed Nov. 30, 2007, which disclosures are herein incorporated by reference in their entirety.

The present invention relates to the storage of items of information in nucleic acid sequences. The invention also relates to nucleic acid sequences in which desired items of information are contained, and to the design, production or use of such sequences.

Important information, especially secret information, must be protected against unauthorized access. To this end, increasingly elaborate cryptographic or steganographic techniques have been developed in the past. Numerous algorithms exist for encrypting data and for disguising secret information. The security of secret steganographic information is based, inter alia, on the fact that its existence is not obvious to an unauthorized person. The information is packaged in an unobtrusive medium, wherein the medium can in principle be selected at will. By way of example, it is known in the prior art to conceal information in digital images or audio files. One pixel of a digital RGB image consists of 3×8 bits. Each 8 bits encode the brightness of the red, green and blue channel. Each channel can accommodate 256 brightness levels. If the last bit (least significant bit, LSB) of each pixel and channel is overwritten with a foreign item of information, the brightness of each channel thus changes by only 1/256, that is to say by 0.4%. To an observer, the image remains unchanged in appearance.

Music on a CD is digitized at 44100 samples/second, 2 channels, 16 bits/sample. When the LSB of a sample is overwritten, the wave amplitude at this point changes by 1/65536, that is to say by 0.002%. This change is inaudible to humans. A conventional CD thus offers space for 74 min×60 sec×44100 samples×2 channels=392 Mbits or ˜50 Mbytes.

In addition, steganographic approaches based on DNA have been developed in recent years. Clelland et al. (Nature 399:533-534 and U.S. Pat. No. 6,312,911), inspired by the microdots used in the Second World War, developed a method for concealing messages in so-called DNA microdots. They produced artificial DNA strands which were composed of a series of triplets, to each of which a letter or a number was assigned. In order to decode the message, the recipient of the secret information must then know the primers for amplification and sequencing as well as the decryption code.

U.S. Pat. No. 6,537,747 discloses methods for encrypting information consisting of words, numbers or graphic images. The information is incorporated directly into nucleic acid strands which are sent to the recipient who can decode the information using a key.

The methods described by Clelland and in U.S. Pat. No. 6,537,747 are based in each case on the direct storage of information in DNA. However, the disadvantage of such direct storage via a simple triplet code is that in this way conspicuous sequence motifs may arise which could be noticed by third parties. As soon as it has been recognized that secret information is contained in a medium, there is a risk that this information will also be decrypted. Furthermore, such DNA domains can perform a biologically relevant function only to a very limited extent. When producing genetically modified organisms, the nucleic acids which contain the encrypted message must therefore be introduced in addition to the genes which bring about the desired characteristics of the organism.

The object of the present invention was therefore to provide an improved steganographic method for embedding information in nucleic acids, which is even more secure against undesired decryption. The information should be concealed in such a way that a third party cannot recognize that any secret information is contained at all.

The inventors of the present invention have discovered that the degeneracy of the genetic code can be used to embed items of information in coding nucleic acids. The degeneracy of the genetic code is understood to mean that a specific amino acid can be encoded by different codons. A codon is defined as a sequence of three nucleobases which encodes an amino acid in the genetic code. According to the invention, a method has been developed with which nucleic acid sequences are provided which are modified in such a way that a desired item of information is contained.

In a first aspect, the subject matter of the invention is a method for designing nucleic acid sequences in which items of information are contained, which comprises the steps:
(a) assigning a first specific value to at least one first nucleic acid codon from a group of degenerate nucleic acid codons which encode the same amino acid,
   assigning a second specific value to at least one second nucleic acid codon from the group,
   optionally assigning one or more further specific values to in each case at least one further nucleic acid codon from the group,
   in which the first and second and optionally further values are in each case allocated at least once within the group of codons which encode the same amino acid;
(b) providing an item of information to be stored as a series of n values which are in each case selected from first and second and optionally further values, in which n is an integer≥1;
(c) providing a starting nucleic acid sequence, wherein the sequence comprises n degenerate codons to which first and second and optionally further values are assigned according to (a), in which n is an integer≥1; and
(d) designing a modified sequence of the nucleic acid from (c), in which, at the positions of the n degenerate codons of the starting nucleic acid sequence, in each case one nucleic acid codon is selected from the group of degenerate codons which encode the same amino acid, to which codon there corresponds a value due to the assignment from (a) so that the series of the values assigned to the n codons results in the item of information to be stored.

A total of 64 different codons are available in the genetic code, which encode in total 20 different amino acids and stop. (Even stop codons are in principle suitable for accommodating information.) A plurality of codons are therefore used for some amino acids and for stop. By way of example, the amino acids Tyr, Phe, Cys, Asn, Asp, Gln, Glu, His and Lys are in each case two-fold encoded. In each case three degenerate codons exist for the amino acid Ile and for stop. The amino acids Gly, Ala, Val, Thr and Pro are in each case four-fold encoded, and the amino acids Leu, Ser and Arg are in each case six-fold encoded. The different codons which encode the same amino acid generally differ only in one of the three bases. Usually, the codons in question differ in the third base of a codon.

In step (a) of the method according to the invention, this degeneracy of the genetic code is used to assign specific values to degenerate nucleic acid codons within a group of codons which encode the same amino acid. In step (a), within a group of degenerate nucleic acid codons which encode the same amino acid, a first specific value is assigned to at least one first nucleic acid codon and a second specific value is assigned to at least one second nucleic acid codon from this group. The first and second values are in each case allocated at least once within the group of codons which encode the same amino acid.

This assignment may take place for one or more of the multi-encoded amino acids. In principle, such an assignment may take place for all of the multi-encoded amino acids. Preferably, an assignment takes place only for the at least three-fold, preferably at least four-fold, more preferably six-fold encoded amino acids. According to the invention, it is particularly preferred to assign specific values only to the codons of the four-fold encoded amino acids and/or to the codons of the six-fold encoded amino acids.

If the two-fold encoded amino acids are also included in the assignment in step (a), only an assignment of a first and a second value can take place. If only the at least four-fold encoded amino acids are included, then in total up to four different values may be allocated within a group of degenerate nucleic acid codons which encode the same amino acid. If only six-fold encoded amino acids are included, then up to six different values may be allocated within a group of degenerate nucleic acid codons.

By the assignment of more than two, i.e. in particular of four or six, different values within a group, a larger quantity of information can be stored via a shorter series of codons. In one embodiment according to the invention, therefore, it is provided in step (a) to assign values only to the codons of those amino acids which are at least four-fold, preferably six-fold encoded. Within the group of degenerate nucleic acid codons which encode the same multi-encoded amino acid, preferably first and second and one or more further values are then assigned to in each case at least one nucleic acid codon from the group. The first and second and optionally further values are in each case allocated at least once within the group of codons.

If only the at least four-fold or six-fold encoded amino acids are included in the assignment of step (a), it is alternatively also possible, within a group of degenerate nucleic acid codons which encode the same amino acid, to assign a first specific value to more than a first nucleic acid codon, i.e. to two, three, four or five nucleic acid codons, and/or to assign a second specific value to more than a second nucleic acid codon from the group, i.e. to two, three, four or five nucleic acid codons. Preferably, the first and second values are in each case allocated multiple times, preferably an equal amount of times, within the group of degenerate codons. In other words, within a group of degenerate nucleic acid codons which encode the same four-fold encoded amino acid, preferably a first value is assigned to two nucleic acid codons and a second value is assigned to two other codons. Correspondingly, if six-fold encoded amino acids are included, preferably a first value is assigned to three nucleic acid codons from a group and a second value is assigned to three other nucleic acid codons which encode the same amino acid. In this way, at least two possible codons which encode the same amino acid are available for each first and for each second value. The alternative of multiple possible codons for one specific value makes it possible to avoid undesired sequence motifs.

In one preferred embodiment of the invention, in step (a) one specific value is assigned to all the nucleic acid codons from a group of degenerate nucleic acid codons which encode the same amino acid. However, it is also possible according to the invention to assign a value to only some of the degenerate nucleic acid codons and not to take account of other nucleic acid codons which encode the same amino acid.

In step (b) of the method according to the invention, an item of information to be stored is provided as a series of n values which are in each case selected from first and second and optionally further values. Here, n is an integer≥1. The item of information to be stored may be, for example, graphic, text or image data. The item of information to be stored may be provided in step (b) in any manner as a series of n values. Care must be taken to ensure that the n values are selected from the same first and second and optionally further values that are assigned to specific nucleic acid codons in step (a). If, therefore, for example only first and second values are assigned in step (a), the item of information to be stored must be provided in step (b) as a series of values which are selected from these first and second values. The item of information to be stored is thus provided in binary form. To this end, text data for example may be represented in binary form by means of the ASCII code, which is known in the field. If, in addition to the first and second values, also one or more further values are assigned in step (a), the item of information to be stored may be provided in step (b) as a series of n values which are selected from first and second and these further values.

In one preferred embodiment, the item of information to be stored is not directly converted into a series of n values, but rather is encrypted beforehand in any known manner. Only the encrypted item of information is then converted into a series of n values as described above.

A starting nucleic acid sequence is provided in step (c) of the method according to the invention. The starting nucleic acid sequence can be selected at will. By way of example, the nucleic acid sequence of a naturally occurring polynucleotide may be used. According to the invention, the term “polynucleotide” is understood to mean an oligomer or polymer composed of a plurality of nucleotides. The length of the sequence is in no way limited by the use of the term polynucleotide, but rather comprises according to the invention any number of nucleotide units. With particular preference, according to the invention, the starting nucleic acid sequence is selected from RNA and DNA. By way of example, the starting nucleic acid may be a coding or non-coding DNA strand. The starting nucleic acid sequence is particularly preferably a naturally occurring coding DNA sequence which encodes a specific protein.

The starting nucleic acid sequence comprises n degenerate codons, to which first and second and optionally further values are assigned according to (a). n is an integer≥1 and corresponds to the number of n values of the item of information to be stored from step (b). The n degenerate codons may optionally be arranged immediately one after the other in the starting nucleic acid sequence or the series thereof may be interrupted by other non-degenerate codons or degenerate codons to which no value is assigned according to (a). Furthermore, it is possible that the series of the n degenerate codons is interrupted at one or more points by non-coding domains. In one preferred embodiment, the n degenerate codons are contained in an uninterrupted coding sequence. With particular preference, the starting nucleic acid encodes a specific polypeptide.

In step (d) of the method according to the invention, a modified sequence of the nucleic acid sequence from (c) is designed. In the modified sequence, at the positions of the n degenerate codons of the starting nucleic acid sequence, in each case nucleic acid codons are selected from the group of degenerate codons which encode the same amino acid, to which codons a value has been assigned due to the assignment from (a). The degenerate codons are selected in such a way that the series of the values assigned to the n codons results in the item of information to be stored.

If the starting nucleic acid sequence encodes a polypeptide, the modified sequence designed in step (d) preferably encodes the same polypeptide. According to the invention, the term “polypeptide” is understood to mean an amino acid chain of any length.

In one embodiment according to the invention, the start and/or end of an item of information can be marked in the modified sequence from step (d) by incorporating an agreed stop sign. By way of example, the series of n codons which result in the item of information to be stored may be followed by a series of several codons to which the same value is assigned.

In one particularly preferred embodiment, the assignment of a first or second or optionally further value to a nucleic acid codon within the group of degenerate codons which encode the same amino acid takes place in step (a) in a manner dependent on the frequency of use of the codon in a specific organism. Different values may be assigned to different degenerate codons on the basis of a species-specific Codon Usage Table (CUT). By way of example, within a group of degenerate nucleic acid codons which encode the same amino acid, a first value may be assigned to the first-best codon, that is to say to the codon used most frequently by a species, and a second value may be assigned to a second-best codon. If only the at least four-fold or six-fold encoded amino acids are included in the assignment of step (a), one or more further values may be allocated in this way within the group of degenerate codons which encode the same amino acid. In one preferred embodiment, only first and second values are allocated within the group. By way of example, in one embodiment, a first value is assigned to the first and the third-best codon and a second value is assigned to the second and the fourth-best codon. Any types of assignment are possible according to the invention, as long as at least a first and at least a second value is assigned within a group of degenerate codons which encode the same amino acid.

Due to the alternative of a plurality of possible codons per value within a group of degenerate codons, it is possible, when designing a modified sequence in step (d), to avoid undesired sequence motifs.

If two or more codons have the same frequency in a species-specific Codon Usage Table, a further condition is agreed upon for the assignment of values.

As an alternative to the assignment of values on the basis of the frequency of use of a codon within a group of degenerate codons or as a further condition, as mentioned above, an assignment may also take place on the basis of an alphabetic sorting. Numerous other assignment possibilities are also conceivable, and the present invention is not intended to be limited to the assignment based on the frequency of codon use.

In one particularly preferred embodiment of the method according to the invention, the modified nucleic acid sequence designed in step (d) may be produced in a subsequent step (e). The production may take place by any method known in the field. By way of example, a nucleic acid with the modified sequence designed in step (d) may be produced by mutation from the starting sequence of step (c). In particular, according to the invention, a substitution of individual nucleobases is suitable for this purpose. Mutation by insertions and deletions is likewise possible. A nucleic acid with the modified sequence can also be produced synthetically in step (e). Methods for producing synthetic nucleic acids are known to a person skilled in the art.

The method according to the invention leads to a modified nucleic acid sequence in which a desired item of information is contained in encrypted form. The key to this lies in the assignment of step (a). This key must be known to the person to whom the item of information is addressed. By way of example, the key can be sent to the addressee separately at a different point in time.

In one particularly preferred embodiment, the key for the assignment according to (a) may itself be encrypted and stored in a nucleic acid. By way of example, the key may additionally be incorporated in the modified nucleic acid sequence obtained in the method according to the invention or may be incorporated separately in another nucleic acid. The key for the assignment of (a) is generally encrypted using another key. Known prior art methods may in principle be used for this purpose. In order that the key stored in a nucleic acid can be found, it is preferably accommodated at an agreed location, for example immediately downstream of a stop codon, downstream of the 3′ cloning site or the like. It is moreover advantageous also to encrypt the stored key itself with a password so that it is not recognizable as such in the nucleic acid sequence.

The present invention also encompasses a modified nucleic acid sequence which is obtainable by a method according to the invention, and a modified nucleic acid which has this nucleic acid sequence and can be obtained by the method according to the invention. Methods for producing nucleic acids are known to a person skilled in the art. By way of example, the production may take place on the basis of phosphoramidite chemistry, by chip-based synthesis methods or solid-phase synthesis methods. However, any other synthesis methods which are familiar to a person skilled in the art may of course also be used.

The subject matter of the invention is also a vector which comprises a modified nucleic acid according to the invention. Methods for inserting nucleic acids into any suitable vector are known to a person skilled in the art.

The invention further relates to a cell which comprises a modified nucleic acid according to the invention or a vector according to the invention, and to an organism which comprises a nucleic acid according to the invention, a cell or a vector according to the invention.

In a further embodiment, the present invention relates to a method for sending a desired item of information, in which a nucleic acid sequence according to the invention, a nucleic acid, a vector, a cell and/or an organism is sent to a desired recipient. Before being sent to the recipient, it is particularly preferred to mix the nucleic acid, the vector, the cell or the organism with other nucleic acids, vectors, cells or organisms which do not contain the desired item of information. These so-called dummies may for example contain no information or may contain other information acting as a diversion and not representing the desired information.

Moreover, the information contained in a nucleic acid sequence modified according to the invention may also serve as a “watermark” for marking a gene, a cell or an organism. In one embodiment, therefore, the subject matter of the invention is the use of a nucleic acid sequence modified according to the invention for labeling a gene, a cell and/or an organism. The marking of genes, cells or organisms with a watermark according to the invention allows them to be clearly identified. The origin and authenticity can thus be clearly established. In order to label a gene, a cell or an organism with a “watermark” according to the invention, a natural nucleic acid sequence of the gene or cell or organism or a portion of the sequence is modified as described above. At the positions of degenerate codons of the starting sequence, codons which encode the same amino acid (or likewise stop) are in each case selected, to which a specific value has been assigned. The codons are selected in such a way that the series of the values assigned thereto in the nucleic acid sequence corresponds to a specific characteristic. This marking cannot be recognized by a third party; the function of the gene, cell or organism is not impaired.

The invention will be further illustrated by the following figures and examples.

### FIGURES

FIG. 1: extract from the international ASCII table.

FIG. 2A: shows the test gene (mouse telomerase) used in Example 1, optimized for H. sapiens

FIG. 2B: shows the encoded protein for the test gene (mouse telomerase) used in Example 1

FIG. 3: Codon Usage Table (CUT) for Homo sapiens

FIG. 4: codon order of the permutations

FIG. 5 shows an analysis of the modified sequence obtained in Example 1 in comparison to the starting sequence

### EXAMPLES

### Example 1

### Encryption of “GENE” in the N-terminus of the Telomerase from M. musculus (Optimized for H. sapiens)

The N-terminus of the telomerase from M. musculus was selected as the carrier for encrypting the message “GENE”. M. musculus telomerase (1251AA) comprises 360 four-fold degenerate, information-containing codons (ICCs) and 372 six-fold degenerate ICCs. The open reading frame (ORF) of the gene is first optimized in a conventional manner, that is to say the codon selection is adapted to the specific circumstances of the target organism.

Hereinbelow, account will be taken only of the codons which are 4-fold and 6-fold degenerate, that is to say for the amino acids VPTAG (4 codons each) and LSR (6 codons each). These are known as ICCs (information-containing codons). (Amino acids for which only 2 or 3 codons exist (DEKNIQHCYF) may in principle also be used. However, since the performance of the gene suffers more severely in this case, they will be disregarded in this example.)

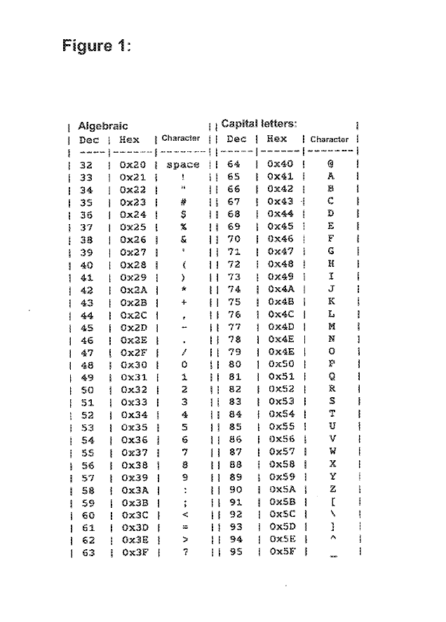

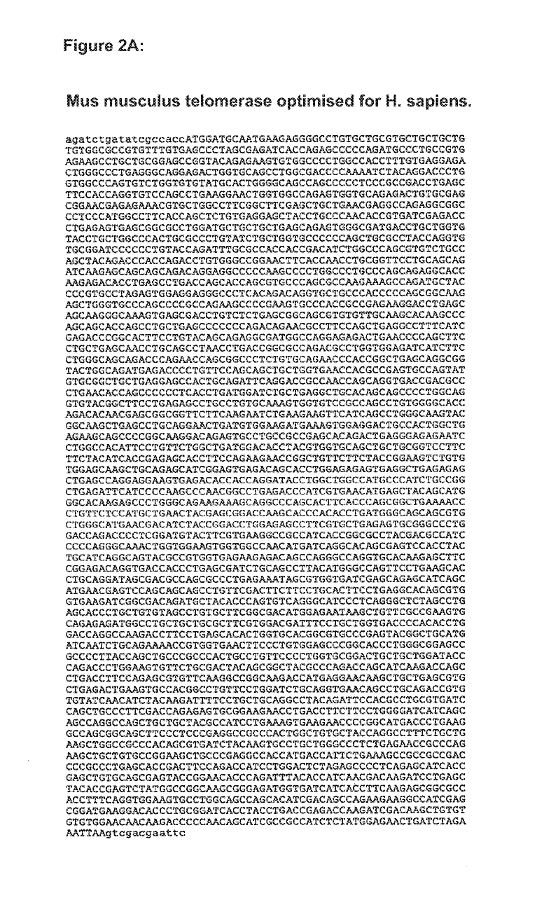

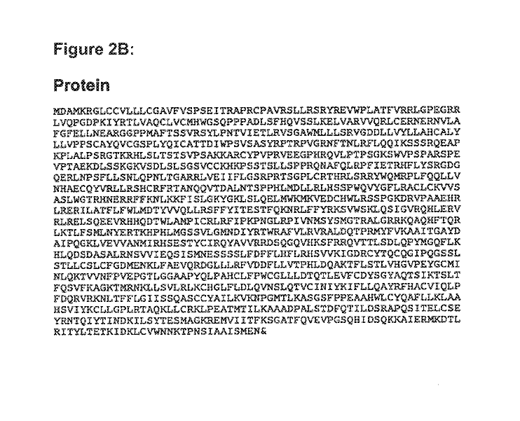

The secret item of information (in some circumstances previously encrypted) is then broken down into bits. Here, 6 bits (=2⁶=64 states) per character are sufficient for letters+numbers+special characters, ideally the ASCII characters from 32=0010 0000 (space) to 95=0101 1111 (underscore). This range includes the capital letters, the numbers and the most important special characters (see FIG. 1). The eight-digit ASCII code is reduced to a 6-bit code using the conventional bit operation: 6 bits=8 bits−32 or 8 bits=6 bits+32.

In this example, the following CUT for Homo sapiens is used for the encryption:

[Key to Figure:

(sortiert nach “Fraction” (1) & alphabetisch (2))=(sorted by “Fraction (1) & alphabetically (2))]

Based on the species-specific Codon Usage Table (CUT), all the ICCs from 5′ to 3′ are then successively modified and the additional information is introduced bit by bit. The following applies:

binary 1=first- or third-best codon
binary 0=second- or fourth-best codon

Here, the “first-” . . . “fourth-best” codon weighting reflects the frequency with which the respective codon is used in the target organism for encoding its amino acid. A database on this subject can be found at: http://www.kazusa.or.jp/codon/.

The alternative of in each case two possible codons per bit makes it possible, most probably in every case, to avoid undesired sequence motifs during the optimization. Of course, ICC-adjacent non-ICC codons can also be modified in order to rule out specific motifs.

A defined CUT is necessary for a clear encryption and decryption. However, especially for little-investigated organisms, CUTs will continue to change in future. In some cases, therefore, it is necessary to deposit a dated CUT. However, only the order of the ICC codons is relevant, not the actual figures relating to the frequency thereof.

The order may be deposited on paper or notarially. Of course, it is also possible to accommodate these data in the DNA itself, for example the 3′ UTR (immediately downstream of the gene). 22 nt are required for depositing the ICC CUT (see Example 2).

However, for the most common target organisms (mammals, crop plants, E. coli, baker's yeast, etc.), the codon tables are so complete that they will not change any further.

If two or more codons have the same frequency in the CUT, the codons in question are sorted alphabetically: A>C>G>T.

The end of a message may be marked by an agreed stop character, for example “11 1111”, corresponding to the underscore character.

The strategy of defining the first- or third-best codon as binary 1 and the second- or fourth-best codon as binary 0, i.e. in general of working with a codon usage table, leads to a gene which is firstly largely optimized and thus functions well in the target organism and secondly permits a watermark.

Alternatively, it is in principle also possible to define as ICCs all the amino acids for which there are two or more codons, and to agree on the following coding principle for steganographic data embedding:

binary 1=G or C at codon position 3
binary 0=A or T at codon position 3

This is possible for the 18 amino acids GEDAVRSKNTIQHPLCYF. (In the above method based on quality ranking, there are only 8 ICCs.) Thus more than twice as much information can be accommodated in a gene and a clear CUT need not be deposited in any case. However, the disadvantage of this method is that the resulting gene is not optimized or is barely optimized.

In the present example, the message “GENE” was encrypted in the N-terminus of the telomerase from M. musculus. This message contains 4×6=24 bits.

| | G | | E | | N | | E | |
|---|---|---|---|---|---|---|---|---|
| “GENE” binär | 0100 | 0111 | 0100 | 0101 | 0100 | 1110 | 0100 | 0101 |
| 8 bit: | (71) | | (69) | | (78) | | (69) | |
| 8 bit-32: | (39) | | (37) | | (46) | | (37) | |
| “GENE” binär | 10 | 0111 | 10 | 0101 | 10 | 1110 | 10 | 0101 |
| 6 bit: | | | | | | | | |

[Key to figure: binär = binary]

In order to encrypt 24 bits, 10 four-fold or six-fold degenerate ICCs were modified in the N-terminus of the telomerase:

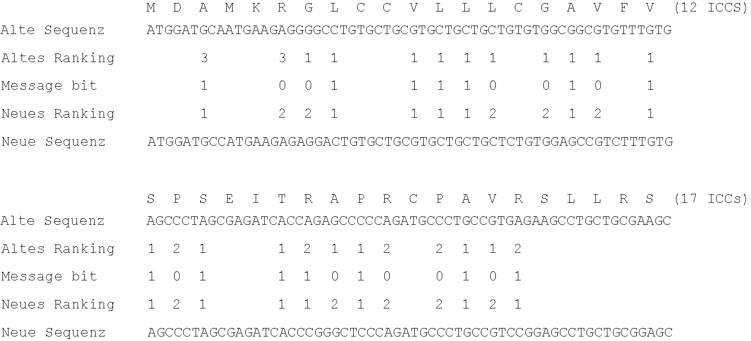

[Key to figure:
Alte Sequenz = Old sequence
Altes Ranking = Old ranking
Neues Ranking = New ranking
Neue Sequenz = New sequence]

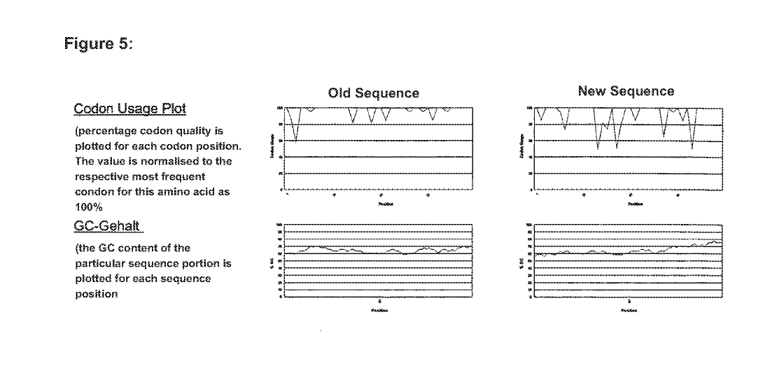

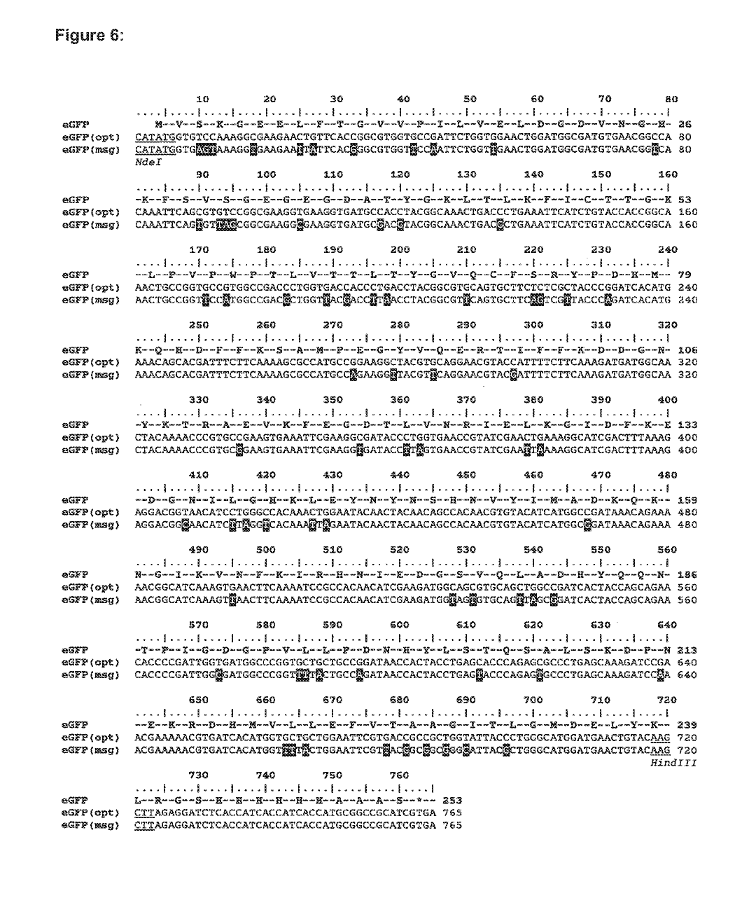

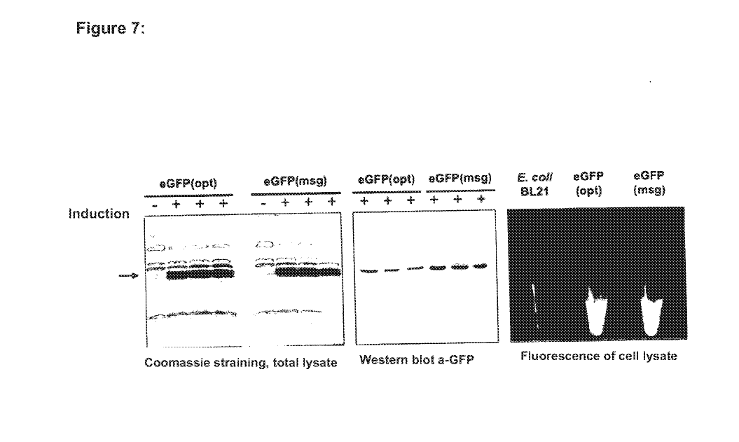

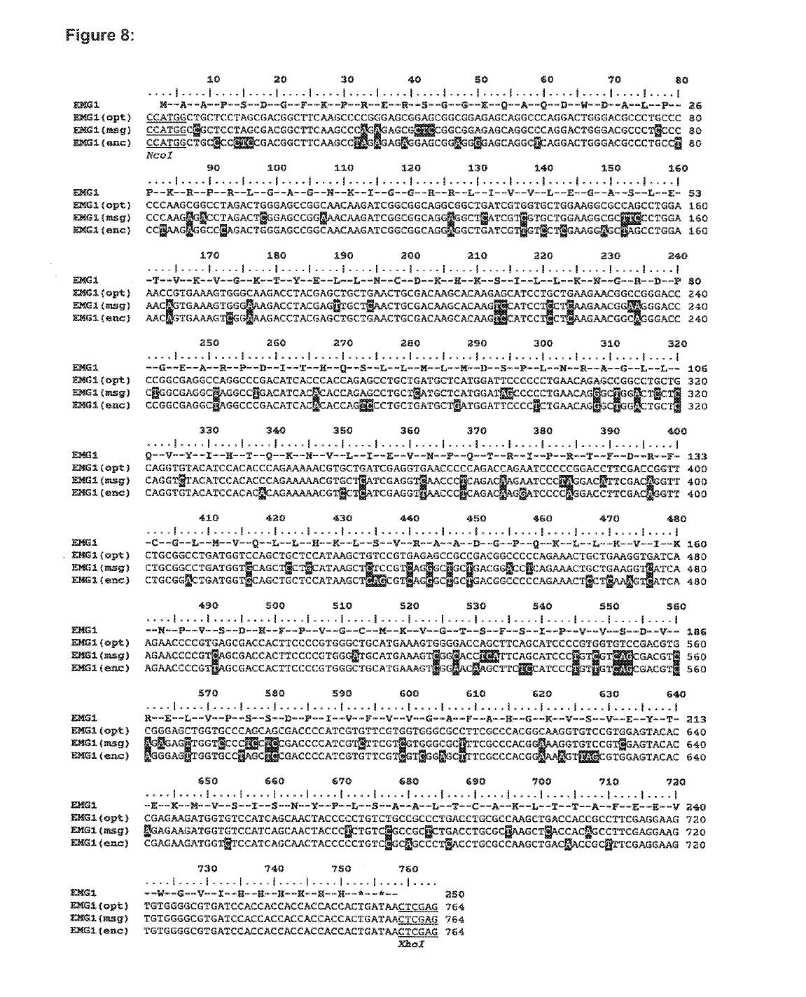

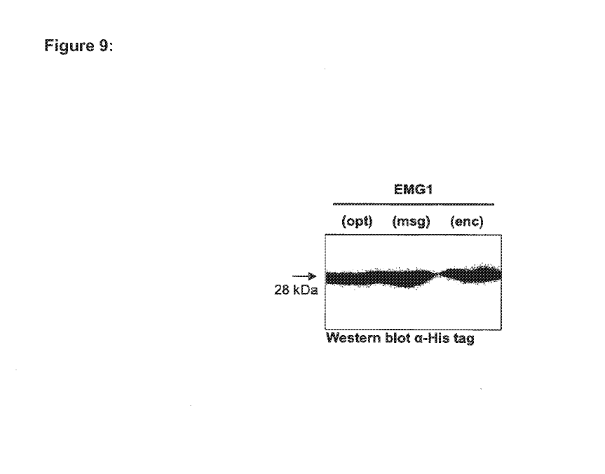

No unwanted motifs or an excessively high GC content occurred during the coding. It was therefore not necessary to make use of the third-best and fourth-best codons. A comparison of the analysis of the starting sequence and of the modified sequence is shown in FIG. 5.

### Example 2

### Depositing a CUT in 22NT

The CUT for Homo sapiens that was used for the encryption in Example 1 was itself encrypted and deposited as a nucleic acid.

First, each codon for an amino acid is given a number (#) which represents its alphabetic position within this group.

Then the ICC CUT is sorted according to the following scheme: 4-fold and 6-fold ICCs->amino acid alphabetically->codon frequency->codon alphabetically

ICC CUT H. sapiens (sorbant nach “Fraction” (1) & alphabetisch (2))

| AA | Cod. | # | Fract. | AA | Cod. | # | Fract. | AA | Cod. | # | Fract. | AA | Cod. | # | Fract. |
|---|---|---|---|---|---|---|---|---|---|---|---|---|---|---|---|
| A | GCC | 2 | 0.40 | L | CTG | 3 | 0.40 | T | ACC | 2 | 0.36 | R | Cod | 5 | 0.21 |
| A | GCT | 4 | 0.28 | L | CTC | 2 | 0.20 | T | ACA | 1 | 0.28 | R | AGA | 1 | 0.20 |
| A | GCA | 1 | 0.23 | L | CIT | 4 | 0.13 | T | ACT | 4 | 0.24 | R | AGG | 2 | 0.20 |
| A | GCG | 3 | 0.31 | L | CIA | 1 | 0.08 | T | ACG | 3 | 0.11 | R | CGC | 4 | 0.19 |
| G | GGC | 2 | 0.34 | P | CCC | 2 | 0.33 | V | GTG | 3 | 0.46 | R | CGA | 3 | 0.11 |
| G | GGA | 1 | 0.25 | P | CCT | 4 | 0.28 | V | GTC | 2 | 0.24 | R | CGT | 5 | 0.08 |
| G | GGG | 3 | 0.25 | P | CCA | 1 | 0.27 | V | GTT | 4 | 0.14 | S | AGC | 1 | 0.24 |
| G | GGT | 4 | 0.16 | P | CCG | 3 | 0.11 | V | GTA | 1 | 0.12 | S | TCC | 4 | 0.22 |
| | | | | | | | | | | | | S | TCY | 6 | 0.18 |
| | | | | | | | | | | | | S | AGT | 2 | 0.15 |
| | | | | | | | | | | | | S | TCA | 3 | 0.15 |
| | | | | | | | | | | | | S | TCG | 5 | 0.06 |

[Key to figure: (sortiert nach “Fraction” (1) & alphabetish (2)) = (sorted by “Fraction (1) & alphabetically (2))]

Each nucleobase is moreover assigned a value and expressed in ASCII code:
A=0 (00)
C=1 (01)
G=2 (10) p1 T=3 (11)

Method 1:

A straight-forward approach is then firstly to list the wobble positions (bold). For the six-fold degenerate ICCs, the rank of the AGN codons of Arg and Ser are additionally shown (underlined).

Here, these AGN ranks are: 2, 3, 1, 4.
Or in binary form:
0010 0011 0001 0100
The first 0 can be omitted (since there is no 8):
010 011 001 100
Translated into nucleotides, this is:
C A T A T A
This CUT accordingly reads:
CTAG CAGT GCTA CTAG CATG GCTA GAGCAT CCTTAG
CATATA

However, it has a length of 42 nt!

The underlined nts are redundant and can be omitted:

CTA CAG GCT CTA CAT GCT GAGCA CCTTA CATATA

This results in a length of just 34 nt.

Method 2:

The length can be further reduced.

Four-fold degenerate ICCs have 4×3×2×1=24, six-fold degenerate ICCs have 6×5×4×3×2×1=720 possible combinations/states.

First, the possible codon orders are sorted and converted into a number.

1234=00, 1243=02, . . . , 4321=23 and . . .
123456=000, . . . , 654321=719 (for the 6-fold ICCs);

| | | |
|---|---|---|
| AA: | Ala Gly Leu Phe Thr Val Arg | Ser |
| Reihenfolge: | 2413 2134 3241 2413 2143 3241 512436 | 146235 |
| In Zahlen: | 10 06 15 10 07 15 515 | 223 |
| Binär | 01011 00110 01111 01010 00111 01111 1000000011 | 0011011111 |
| In nt | C C G C G C T G G G ATGTT GAAAT | ATCTT |
| Nochmal | CCGCGCTGGGATGTTGAAATATCTT | |

[Key to figure:
Reihenfolge = Order
In Zahlen = In number form
Binär = In binary form
Nochmal = Again]

Thus: 6×2.5+2×5=25 nt are required.

(However, this range can then embrace all states between poly(A) & (fast)poly(T).)

In order that the deposited CUT can be found, it should be accommodated at an agreed location (for instance immediately downstream of the stop codon, downstream of the 3′ cloning site or the like)—optionally flanked by clear sequence motifs or primer binding sites).

Moreover, the deposited ICC CUT may also be encrypted with a password, so that it is not recognizable as such.

1.-26. (canceled)

27. A method for producing an information containing nucleic acid molecule, the method comprising the steps:
(a) selecting a starting nucleic acid molecule for the incorporation of the items of information;
(b) selecting codons of the starting nucleic acid molecule that may be altered to incorporate the information;
(c) altering the nucleotide sequence to incorporate the information, thereby generating the nucleotide sequence of the information containing nucleic acid molecule; and
(d) producing the information containing nucleic acid molecule based upon the sequence generated in step (c);
wherein the information containing nucleic acid molecule encodes a protein,
wherein incorporation of the message does not change the amino acid sequence of the encoded protein,
wherein the only codons altered to incorporate the information and read to disclose the information are codons for the following eight amino acids: arginine, valine, glycine, alanine, threonine, serine, leucine, and proline,
wherein the encoded information is read from 5′ to 3′ and each codon encoding the eight amino acids is read as a zero or one,
wherein a set of zeros and ones represents a character of information, and
wherein expression level of the encoded protein in a human cell is not measurably decreased for the information containing nucleic acid molecule compared to the starting nucleic acid molecule.

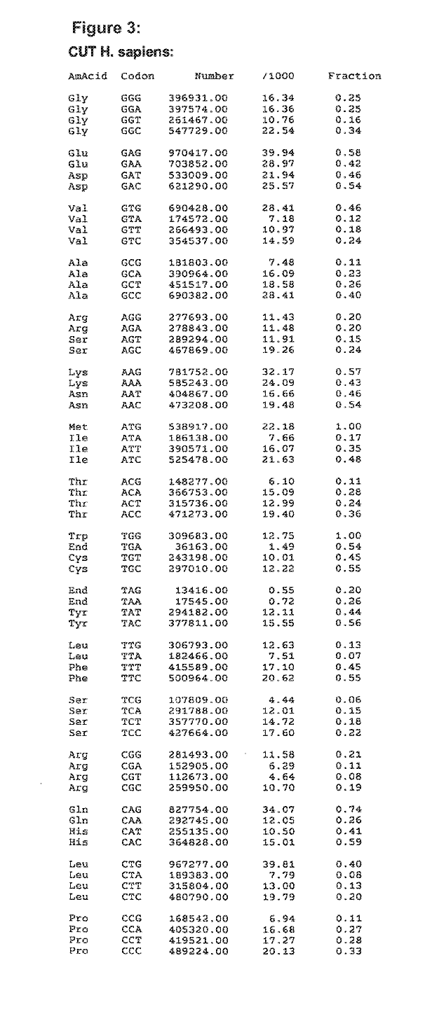

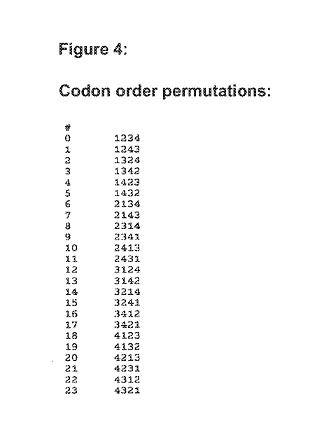

28. The method of claim 27, wherein (i) the most prevalent codon in FIG. 3 for each of the eight amino acids is read as a zero and the second most prevalent codon in FIG. 3 for each of the eight amino acids is read as a one or (ii) the most prevalent codon in FIG. 3 for each of the eight amino acids is read as the second most prevalent codon in FIG. 3 for each of the eight amino acids is read as a zero.

29. The method of claim 27, wherein more than codon is selected to represent a zero and more than one codon is selected to represent a one.

30. The method of claim 29, wherein codons selected to represent zeros and ones alternate based upon codon usage preference for a particular organism.

31. The method of claim 30, wherein the codons for serine are read as the digits of either zero or one, wherein (i) AGC, TCT, and AGT are each read as a zero and TCC, TCA, and TCG are each read as a one or (ii) AGC, TCT, and AGT are each read as a one and TCC, TCA, and TCG are each read as a zero.

32. The method of claim 30, wherein the first most preferred codon encoding for an amino acid is read as a zero and the second most preferred amino acid is read as a one.

33. The method of claim 30, wherein the first most preferred codon encoding for the amino acid serine is AGC and the second most preferred codon encoding for the amino acid serine is TCC.

34. The method of claim 30, wherein the third most preferred codon encoding for an amino acid is read as a one and the fourth most preferred amino acid is read as a zero.

35. The method of claim 34, wherein the third most preferred codon encoding for the amino acid serine is TCT and the fourth most preferred codon encoding for the amino acid serine is TCA.

36. The method of claim 27, wherein the starting nucleic acid molecule is codon optimized for a particular organism.

37. The method of claim 27, wherein the zeros and ones are read in groups of six or eight to represent a single character.

38. The method of claim 37, wherein the six digit binary code 100111 represents the following character: G.

39. The method of claim 27, wherein the information containing nucleic acid molecule produced in step (d) is a linear nucleic acid molecule.

40. The method of claim 27, wherein the information containing nucleic acid molecule produced in step (d) is contained in a vector.

41. A method for producing an information containing nucleic acid molecule, the method comprising the steps:
(a) generating the nucleotide sequence of the information containing nucleic acid molecule; and
(b) producing the information containing nucleic acid molecule based upon the sequence generated in step (a);
wherein the information containing nucleic acid molecule encodes a protein,
wherein the only codons that are read to disclose the information are codons for the following eight amino acids: arginine, valine, glycine, alanine, threonine, serine, leucine, and proline,
wherein the encoded information is read from 5′ to 3′ and each codon of the eight amino acids is read as a zero or one,
wherein (i) the most prevalent codon in FIG. 3 for each of the eight amino acids is read as a zero and the second most prevalent codon in FIG. 3 for each of the eight amino acids is read as a one or (ii) the most prevalent codon in FIG. 3 for each of the eight amino acids is read as the second most prevalent codon in FIG. 3 for each of the eight amino acids is read as a zero,
wherein a set of zeros and ones represents a character of information.

42. The method of claim 41, wherein expression level of the encoded protein in a human cell is not measurably decreased for the information containing nucleic acid molecule compared to a fully codon optimized nucleic acid molecule encoding the identical amino acid sequence.

43. The method of claim 41, wherein the information containing nucleic acid molecule is codon optimized for a particular organism.
